(19) United States
(12) Patent Application Publication
(10) Pub. No.: US 2019/0070154 A1
(43) Pub. Date: Mar. 7, 2019

(54) NEW METHODS OF USE FOR AN ANTI-DIARRHEA AGENT

(71) Applicant: Duke University, Durham, NC (US)

(72) Inventors: Wei Chen, Chapel Hill, NC (US); Robert A. Mook, Jr., Chapel Hill, NC (US); Jiangbo Wang, Durham, NC (US); Xiu-rong Ren, Durham, NC (US); Minyong Chen, Durham, NC (US); Lawrence S. Barak, Durham, NC (US); Herbert Kim Lyerly, Chapel Hill, NC (US)

(21) Appl. No.: 15/739,396

(22) PCT Filed: Jun. 24, 2016
(86) PCT No.: PCT/US2016/039213

§ 371 Date: Dec. 22, 2017

Related U.S. Application Data
(60) Provisional application No. 62/184,123, filed on Jun. 24, 2015.
(60) Provisional application No. 62/193,944, filed on Jul. 17, 2015.

(51) Int. Cl. A61K 31/44; A61K 45/06; A61K 31/426; A61K 39/395; A61K 33/24; A61K 31/282

(52) U.S. Cl. CPC A61K 31/44; A61K 45/06; A61K 31/426; A61K 39/3955; A61K 33/24; A61K 31/282; A61K 39/39558

(57) ABSTRACT

Described are methods of treating a disease associated with dysregulation of the Wnt/Frizzled signaling pathway. The methods include identifying subjects in need of therapy, administering inhibitors of the Wnt/Frizzled signaling pathway, pharmaceutical compositions including the inhibitors, and methods of using the compounds and compositions for treating cancer, bacterial Sand viral infection, lupus, type II diabetes, nonalcoholic steatohepatitis (NASH) and nonalcoholic fatty liver disease (NAFLD) in a subject.

### CROSS-REFERENCE TO RELATED APPLICATIONS

This application claims priority to U.S. Provisional Patent Application No. 62/184,123, filed on Jun. 24, 2015, and to U.S. Provisional Patent Application No. 62/193,944, filed on Jul. 17, 2015, the contents of each of which are hereby incorporated by reference in their entireties.

### STATEMENT OF GOVERNMENT INTEREST

This invention was made with government support under Grant numbers R01-CA172570 awarded by the National Cancer Institute (NCI); 5K12-CA100639 awarded by the National Cancer Institute (NCI); and BC123289 awarded by the Department of Defense (DOD). The government has certain rights in the invention.

### TECHNICAL FIELD

The present disclosure relates to compounds, compositions, and methods for treating Wnt/Frizzled related diseases and/or disorders, such as cancer.

### BACKGROUND

Wnt proteins are secreted glycoproteins that bind and activate the seven transmembrane receptor Frizzled and single transmembrane receptors LRP5/6. Wnt binding to Frizzled and LRP5/6 results in activation of cytosolic proteins Dishevelled (Dvl), leading to internalization of the Frizzled receptor. Downstream signaling events resulting from Wnt binding include the stabilization and translocation of cytosolic β-catenin proteins into the nucleus, activation of the transcription factor LEF/TCF and transcription of Wnt/β-catenin target genes.

The Wnt signaling pathway plays a key role in tissue development and homeostasis and is dysregulated in many diseases including cancer. For example, in colorectal cancer (CRC) more than 80% of all sporadic and hereditary cancers show hyperactivation of the pathway due to mutations in the adenomatous polyposis coli (APC) or the β-catenin gene. Given the importance of the Wnt signaling activity underlying tumor formation and metastasis, there exists a need for therapies that inhibit the Wnt signaling pathway.

### SUMMARY OF THE INVENTION

In one aspect, disclosed is a method of treating a disease associated with dysregulation of the Wnt/Frizzled signaling pathway in a subject in need thereof, the method comprising administering to the subject an effective amount of a compound of formula (I), or a pharmaceutically acceptable salt thereof,

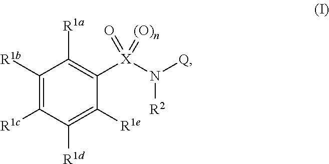

wherein, one of R^{1a}, R^{1b}, R^{1c}, R^{1d} and R^{1e} is OR⁴ or NR⁸—SO₂—R⁹, and the remaining are independently selected from hydrogen, halogen, nitro, alkyl, cyano, haloalkyl, alkoxyalkyl, heteroalkyl, alkenyl, alkynyl, heterocycle, carboxyl, heterocyclealkyl, OR⁴, SR⁵, NR⁶R⁷ and NR⁸—SO₂—R⁹; or R^{1b} and R^{1c}, R^{1c} and R^{1d}, or R^{1d} and R^{1e} together form a six-membered aromatic ring; X is C or S; n is 0 or 1; R² is selected from hydrogen, —C(O)-alkyl, —C(O)— alkenyl, —C(O)-alkoxyalkyl, —C(O)-heteroalkyl, —C(O)-heteroaryl, —C(O)—O-heteroalkyl, —C(O)—O-heteroaryl, —C(O)—O-alkyl, —C(O)—O-alkenyl, and —C(O)—O-alkoxyalkyl, or R² and R^{1e} together form a ring; Q is heteroaryl, with 0-5 substituents independently selected from hydrogen, halogen, nitro, alkyl, cyano, haloalkyl, alkoxyalkyl, heteroalkyl, alkenyl, alkynyl, heterocycle, carboxyl, heterocyclealkyl, OR⁴, SR⁵, NR⁶R⁷, and NR⁸—SO₂—R⁹; R⁴ is selected from hydrogen, —C(O)-alkyl, —C(O)-alkenyl, —C(O)-alkoxyalkyl, —C(O)-heteroalkyl, —C(O)— heteroaryl, —C(O)—O-heteroalkyl, —C(O)—O-heteroaryl, —C(O)—O-alkyl, —C(O)—O-alkenyl, and —C(O)—O-alkoxyalkyl —C(O)—NH-alkyl, and —C(O)-heterocycle; R⁵, R⁶ and R⁷ are each independently selected from hydrogen, alkyl, —C(O)-alkyl, —C(O)-alkoxyalkyl, alkenyl, alkynyl, and heteroalkyl; R⁸ is selected from hydrogen and alkyl; and R⁹ is selected from hydrogen, alkyl, aryl, heteroaryl, arylalkyl, heterocycle, and heteroarylalkyl.

In another aspect, disclosed are methods for treating cancer in a subject in need thereof, the method comprising identifying a subject with dysregulated Wnt/Frizzled signaling pathway; and administering to the subject with dysregulated Wnt/Frizzled signaling pathway effective amount of a compound of formula (I) or a pharmaceutically acceptable salt thereof to a subject in need thereof.

In another aspect, disclosed are methods of modulating the Wnt/Frizzled signaling pathway in a subject, the method comprising administering to the subject an effective amount of a compound of formula (I) or a pharmaceutically acceptable salt thereof to a subject in need thereof.

In another aspect, disclosed are pharmaceutical compositions comprising at least one pharmaceutically acceptable carrier and an effective amount of a compound of formula (I).

### DETAILED DESCRIPTION

Disclosed herein are methods of treating a disease associated with dysregulation of the Wnt/Frizzled signaling pathway. The Wnt/Frizzled signaling pathway has been implicated in a number of different diseases and/or disorders such as cancer and metabolic diseases such as type II diabetes. Based on the multifunctional bioactivity of the Wnt inhibitor niclosamide, this pathway may also be implicated in other diseases and/or disorders such as bacterial and viral infection, lupus, nonalcoholic steatohepatitis (NASH) and nonalcoholic fatty liver disease (NAFLD). Treatment of the disease associated with dysregulation of the Wnt/Frizzled signaling pathway may be accomplished by use of the compounds disclosed herein.

In particular, the known anti-diarrhea agent, nitazoxanide, was discovered to be an inhibitor of the Wnt/Frizzled signaling pathway. As a result, compounds based on the nitazoxanide chemotype were synthesized and evaluated for Wnt inhibition. Accordingly, the compounds disclosed herein are inhibitors of the Wnt/Frizzled signaling pathway.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

A “disease associated with dysregulation of the Wnt/Frizzled signaling pathway,” as used herein, is a disease in which the Wnt/Frizzled signaling pathway is dysregulated. Certain exemplary Wnt/Frizzled-related diseases include, but are not limited to, cardiovascular disease, neoplasm, obesity, osteoporosis, neuron degeneration, cancer, diabetes and disorders in wound healing and tissue repair. The Wnt/Frizzled signaling pathway may be considered dysregulated when, for example, diseased tissue and/or cells comprise at least one of: increased levels of β-catenin; increased LEF/TCF-mediated transcription; increased levels of one or more Wnt proteins, including, but not limited to, Wnt3A; increased levels of Frizzled; and/or increased levels of Dishevelled; as compared to normal tissue and/or cells. As used herein, the term “tissue” includes all biological tissues, including, but not limited to, organ tissue, tumor tissue, skin, blood, etc.

The term “effective amount,” as used herein, refers to a dosage of the compounds or compositions effective for eliciting a desired effect. This term as used herein may also refer to an amount effective at bringing about a desired in vivo effect in an animal, preferably, a human, such as treatment of a disease.

The term “treatment”, as used herein in the context of treating a condition, pertains generally to treatment and therapy, whether of a human or an animal (e.g. in veterinary applications), in which a desired therapeutic effect is achieved. For example, treatment includes prophylaxis and can ameliorate or remedy the condition, disease, or symptom, or treatment can inhibit the progress of the condition or disease (e.g., reduce the rate of disease/symptom progression or halt the rate of disease/symptom progression).

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “alkyl” as used herein, means a straight or branched, saturated hydrocarbon chain containing from 1 to 20 carbon atoms. The term “lower alkyl” or “C₁-C₆-alkyl” means a straight or branched chain hydrocarbon containing from 1 to 6 carbon atoms. The term “C₁-C₃-alkyl” means a straight or branched chain hydrocarbon containing from 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl” as used herein, means an unsaturated hydrocarbon chain containing from 2 to 20 carbon atoms and at least one carbon-carbon double bond.

The term “alkynyl” as used herein, means an unsaturated hydrocarbon chain containing from 2 to 20 carbon atoms and at least one carbon-carbon triple bond.

The term “alkoxyalkyl” as used herein, refers to an alkoxy group, as defined herein, appended to the parent molecular moiety through an alkylene group, as defined herein.

The term “arylalkyl” as used herein, refers to an aryl group, as defined herein, appended to the parent molecular moiety through an alkylene group, as defined herein.

The term “heteroarylalkyl” as used herein, refers to a heteroaryl group, as defined herein, appended to the parent molecular moiety through an alkylene group, as defined herein.

The term “heterocyclealkyl” as used herein, refers to a heterocycle group, as defined herein, appended to the parent molecular moiety through an alkylene group, as defined herein.

The term “alkylene”, as used herein, refers to a divalent group derived from a straight or branched chain hydrocarbon of 1 to 10 carbon atoms, for example, of 2 to 5 carbon atoms. Representative examples of alkylene include, but are not limited to, —CH₂CH₂—, —CH₂CH₂CH₂—, —CH₂CH₂CH₂CH₂—, and —CH₂CH₂CH₂CH₂CH₂—.

The term “alkoxy” as used herein, means at least one alkyl group, as defined herein, is appended to the parent molecular moiety through an oxygen atom. Representative examples of alkoxy include, but are not limited to, methoxy, ethoxy, and isopropoxy.

The term “aryl” as used herein, refers to a phenyl group, or a bicyclic fused ring system. Bicyclic fused ring systems are exemplified by a phenyl group appended to the parent molecular moiety and fused to a cycloalkyl group, as defined herein, a phenyl group, a heteroaryl group, as defined herein, or a heterocycle, as defined herein. Representative examples of aryl include, but are not limited to, indolyl, naphthyl, phenyl, quinolinyl and tetrahydroquinolinyl.

The term “carboxyl” as used herein, means a carboxylic acid, or —COOH.

The term “haloalkyl” as used herein, means an alkyl group, as defined herein, in which one, two, three, four, five, six, seven or eight hydrogen atoms are replaced by a halogen. Representative examples of haloalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “halogen” as used herein, means Cl, Br, I, or F.

The term “heteroaryl” as used herein, refers to an aromatic monocyclic ring or an aromatic bicyclic ring system. The aromatic monocyclic rings are five or six membered rings containing at least one heteroatom independently selected from the group consisting of N, O and S. The five membered aromatic monocyclic rings have two double bonds and the six membered six membered aromatic monocyclic rings have three double bonds. The bicyclic heteroaryl groups are exemplified by a monocyclic heteroaryl ring appended to the parent molecular moiety and fused to a monocyclic cycloalkyl group, as defined herein, a monocyclic aryl group, as defined herein, a monocyclic heteroaryl group, as defined herein, or a monocyclic heterocycle, as defined herein. Representative examples of heteroaryl include, but are not limited to, indolyl, pyridinyl (including pyridin-2-yl, pyridin-3-yl, pyridin-4-yl), pyrimidinyl, thiazolyl, and quinolinyl.

The term “heterocycle” or “heterocyclic” as used herein, means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.1^{3,7}]decane), and oxa-adamantane (2-oxatricyclo[3.3.1.1^{3,7}]decane). The monocyclic, bicyclic, and tricyclic heterocycles are connected to the parent molecular moiety through any carbon atom or any nitrogen atom contained within the rings, and can be unsubstituted or substituted.

The term “heteroalkyl,” as used herein, means an alkyl group, as defined herein, in which at least one of the carbons of the alkyl group is replaced with a heteroatom, such as oxygen, nitrogen, and sulfur.

The term “hydroxyl” or “hydroxy” as used herein, means an —OH group.

In some instances, the number of carbon atoms in a hydrocarbyl substituent (e.g., alkyl or cycloalkyl) is indicated by the prefix “Cₓ-C_{y}-”, wherein x is the minimum and y is the maximum number of carbon atoms in the substituent. Thus, for example, “C₁-C₃-alkyl” refers to an alkyl substituent containing from 1 to 3 carbon atoms.

The term “substituents” refers to a group “substituted” on an aryl, heteroaryl, phenyl or pyridinyl group at any atom of that group. Any atom can be substituted.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. METHODS OF TREATMENT

The disclosed compounds and compositions may be used in methods for treatment of Wnt/Frizzled related medical disorders and/or diseases. The methods of treatment may comprise administering to a subject in need of such treatment a composition comprising an effective amount of the compound of formula (I).

The compositions can be administered to a subject in need thereof to modulate the Wnt/Frizzled signaling pathway for a variety of diverse biological processes. The present disclosure is directed to methods for administering the compositions to inhibit the Wnt/Frizzled signaling pathway, a pathway that plays a key role in tissue development and homeostasis and is dysregulated in many diseases including cancer and metabolic diseases such as type II diabetes. Based on the multifunctional bioactivity of the Wnt inhibitor niclosamide, this pathway may also be implicated in other diseases and/or disorders such as lupus, bacterial and viral infection, nonalcoholic steatohepatitis (NASH) and nonalcoholic fatty liver disease (NAFLD). Accordingly, the disclosed compounds and compositions may be administered to a subject for the treatment of cancer, type II diabetes, lupus, bacterial and viral infection, nonalcoholic steatohepatitis (NASH) and nonalcoholic fatty liver disease (NAFLD).

The compositions may be useful for treating and preventing certain diseases and disorders in humans and animals related to Wnt/Frizzled dysfunction. Treatment or prevention of such diseases and disorders can be effected by inhibiting the Wnt/Frizzled signaling pathway in a subject, by administering a compound or composition of the disclosure, either alone or in combination with another active agent as part of a therapeutic regimen to a subject in need thereof.

In certain embodiments, provided are methods of identifying a subject with a disease associated with dysregulation of the Wnt/Frizzled signaling pathway. The methods may comprise determining the level of at least one protein in a sample from a subject, wherein the protein is involved in the Wnt/Frizzled signaling pathway, and comparing the level of the protein to a standard level. An increased level of the protein may be indicative of a subject having a Wnt/Frizzled-related disease.

The methods of treatment may comprise determining the level of at least one protein in a sample from a subject, wherein the protein is involved in the Wnt/Frizzled signaling pathway, and comparing the level of the protein to a standard level, wherein an increased level of the protein may be indicative of a subject having a disease associated with dysregulation of the Wnt/Frizzled signaling pathway, and further administering to the subject an inhibitor of the Wnt/Frizzled signaling pathway.

a. Cancer

Inhibition of the Wnt/Frizzled signaling pathway can lead to treatment and reduction of cancer or tumor growth, and/or reduce metastasis of cancerous or tumor cells. Accordingly, the disclosed compositions can be used in methods that treat and/or prevent cancer or tumors in a subject administered the compound. The method can treat cancer or tumor based growth and can be any type of cancer such as, but not limited to, breast cancer, melanoma, prostate cancer, lung cancer, ovarian cancer, esophageal cancer, glioblastoma, multiple myeloma, mantle cell lymphoma, liver cancer, leukemia, acute myelogenous leukemia, or a combination thereof.

In some embodiments, the administered composition to a subject in need thereof can mediate reduction, clearance or prevention of additional growth of tumor cells by inhibiting the Wnt/Frizzled signaling pathway, thereby reducing growth/proliferation of tumor cells, but does not have an effect on normal cells.

In some embodiments, the administered composition can increase tumor free survival, reduce tumor mass, slow tumor growth, increase tumor survival, or a combination thereof in the subject. The administered composition can reduce tumor volume in the subject in need thereof. The administered composition can increase tumor free survival in the subject after administration of the composition.

In some embodiments, the composition can be administered to clear or eliminate the cancer or tumor expressing the one or more oncogenes without damaging or causing illness or death in the subject administered the composition.

In certain embodiments, a subject in need of treatment for cancer may have at least one inactivating mutation of the Adenomatous Polyposis Coli (APC) gene, which is related to the Wnt/Frizzled signaling pathway. In certain embodiments, a subject in need of treatment for cancer may have at least one mutation of the β-catenin gene or overexpression of the β-catenin protein, or a combination thereof. In certain embodiments, a subject in need of treatment for cancer may have overexpression of Wnt ligands.

In certain embodiments, determining whether a cancer comprises a dysregulated Wnt/Frizzled signaling pathway may comprise detecting the level of one or more of Wnt, Frizzled, β-catenin, and/or Dishevelled, and comparing the level to normal tissue and/or cells. In certain such embodiments, if the cancer comprises higher levels of Wnt, Frizzled, β-catenin and/or Dishevelled as compared to normal tissue and/or cells, the cancer is predicted to respond to treatment with an inhibitor of the Wnt/Frizzled signaling pathway. In certain embodiments, determining whether a cancer comprises a dysregulated Wnt/Frizzled signaling pathway comprises detecting the level of LEF/TCF-mediated transcription as compared to LEF/TCF-mediated transcription in normal tissue and/or cells. In certain such embodiments, if the cancer comprises a higher level of LEF/TCF-mediated transcription as compared to normal tissue and/or cells, the cancer is predicted to respond to treatment with an inhibitor of the Wnt/Frizzled signaling pathway.

A variety of sources (Howe, et al. Cancer Biology and Therapy 2004, 3(1), 36-41; Taketo, M. Nature Genetics 2004, 36, 320-22; Minde et al. PLOS ONE 2013, 8(10), e77257) have reported that activity of the Wnt/Frizzled pathway is involved in the development of benign and malignant breast tumors. Furthermore, its presence is indicated with elevated levels of β-catenin in the nucleus and/or cytoplasm, and increased β-catenin expression is strongly correlated with poor prognosis in breast cancer patients. This accumulation may be due to several factors such as mutations in β-catenin, deficiencies in the β-catenin destruction complex, most frequently by mutations in structurally disordered regions of APC, overexpression of Wnt ligands, loss of inhibitors, and/or decreased activity of regulatory pathways. Breast tumors have also been seen to metastasize due to Wnt involvement in the epithelial-mesenchymal transition (EMT). Investigation of the metastasis of basal-like breast cancer to the lungs has shown that repression of Wnt/β-catenin signaling can prevent EMT, which can inhibit metastasis (DiMeo, et al. Cancer Research 2009, 69(13), 5364-5373).

Wnt signaling has also been implicated in the development of other cancers. Changes in CTNNB1 expression, which is the gene that encodes β-catenin, can be measured in not just breast cancer, but also colorectal cancer, melanoma, prostate cancer, lung cancer, and several other cancer types. Increased expression of Wnt ligand-proteins such as Wnt 1, Wnt2, and Wnt7A have been observed in the development of glioblastoma, esophageal cancer, and ovarian cancer respectively. Other proteins known to cause multiple types of cancer in the absence of proper functioning include ROR1, ROR2, SFRP4, Wnt5A, WIF1, and those of the TCF/LEF family (Anastas, et al. Nature Reviews Cancer 2012, 13 (1), 11-26).

Accordingly, the foregoing firmly implicate the Wnt/Frizzled signaling pathway in the biology of a variety of cancer types and distinguish it as a cancer target.

b. Modes of Administration

Methods of treatment may include any number of modes of administering a disclosed composition. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire™). In the pharmaceutical composition, the agent may also be dispersed in a microparticle, e.g. a nanoparticulate composition.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrastemal, subcutaneous and intraarticular injection and infusion.

c. Combination Therapies

Additional therapeutic agent(s) may be administered simultaneously or sequentially with the disclosed compounds and compositions. Sequential administration includes administration before or after the disclosed compounds and compositions. In some embodiments, the additional therapeutic agent or agents may be administered in the same composition as the disclosed compounds. In other embodiments, there may be an interval of time between administration of the additional therapeutic agent and the disclosed compounds. In some embodiments, administration of an additional therapeutic agent with a disclosed compound may allow lower doses of the other therapeutic agents and/or administration at less frequent intervals. When used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of the present disclosure. The above combinations include combinations of a compound of the present disclosure not only with one other active compound, but also with two or more other active compounds. For example, the compound of the disclosure can be combined with a variety of anti-cancer drugs and chemotherapeutics.

The compound of Formula I can be combined with the following, but not limited to, actinomycins, alkylating agents, anthracyclines, antifolates, antiestrogen agents, anti-metabolites, anti-androgens, antimicrotubule agents, aromatase inhibitors, bleomycins, Ca²⁺ adenosine triphosphate (ATP)ase inhibitors, cytosine analogs, deltoids/retinoids, dihydrofolate reductase inhibitors, deoxyribonucleic acid (DNA) topoisomerase inhibitors, dopaminergic neurotoxins, glucocorticoids, histone deacetylase inhibitors, hormonal therapies, immunotherapeutic agents, inosine monophosphate (IMP) dehydrogenase inhibitors, isoprenylation inhibitors, luteinizing hormone-releasing hormone agonists, mammalian target of rapamycin (mtor) inhibitors, multi-drug resistance (MDR) inhibitors, mitomycins, photodyamic therapies, proteasome inhibitors, platinum containing compounds, radiation, receptor tyrosine kinase inhibitors, ribonucleotide reductase inhibitors, thrombospondin mimetics, uracil analogs, vinca alkaloids, and vitamin D3 analogs. Specific anti-cancer or chemotherapeutic agents that may be combined with a disclosed compound include actinomycin D, AG13736, alisertib, 17-allylamino-17-demethoxygeldanamycin, altretamine, 9-aminocamptothecin, N-(4-(3-amino-1H-indazol-4-yl)phenyl}-N′-(2-fluoro-5-methylphenyl)urea, N-(4-(4-aminothieno[2,3-d]pyrimidin-5-yl)phenyl}-N′-(2-fluoro-5-(trifluoromethyl)phenyl)urea, anastozole, AP-23573, asparaginase, axitinib, azacitidine, bevacizumab, bicalutamide, bevacizumab, bleomycin a2, bleomycin b2, bortezemib, busulfan, campathecins, carboplatin, carmustine (BCNU), CB1093, CHOP (C: Cytoxan® (cyclophosphamide); H: Adriamycin® (hydroxydoxorubicin); O: Vincristine (Oncovin®); P: prednisone), chlorambucil, CHIR258, cilengitide, cisplatin, CNF-101, CNF-1001, CNF-2024, CP547632, crisnatol, cytarabine, cyclophosphamide, cytosine arabinoside, daunorubicin, dabrafenib, dacarbazine, dactinomycin, dasatinib, daunorubicin, deferoxamine, demethoxyhypocrellin A, depsipeptide, 17-dimethylaminoethylamino-17-demethoxygeldanamycin, docetaxel, doxifluridine, doxorubicin, EB 1089, enzastaurin, epothilone D, epirubicin, 5-ethynyl-1-13-D-ribofuranosylimidazole-4-carboxamide (EICAR), erlotinib, etoposide, everolimus, 5-fluorouracil (5-FU), floxuridine, fludarabine, flutamide, gefitinib, geldanamycin, gemcitabine, goserelin, N-(2-(4-hydroxyanilino}-3-pyridinyl}-4-methoxybenzenesulfonamide, hydroxyurea, idarubicin, ifosfamide, imatinab, interferon-a, interferon-y, IPI-504, irinotecan, KH 1060, lapatinib, leucovorin calcium, LAQ824, leuprolide acetate, letrozole, lomustine (CCNU), melphalan, mercaptopurine, methotrexate, 1-methyl-4-phyenylpyridinium, MG132, mitoxantrone, mitozolomide, MLN4924, MLN518, MS-275, mycophenolic acid, nedaplatin, oprelvekin, oxaliplatin, paclitaxel, PD98059, pazopanib, peplomycin, phtalocyanine, pirarubicin, plicamycin, procarbazine, PTK787, PU24FC1, PU3, radicicol, raloxifene, rapamycin, ratitrexed, pheuretinide, ribavirin, rituximab (Rituxin®), satraplatin, sorafenib, staurosporine, suberoylanilide hydroxamic acid, sunitinib, tamoxifen, taxol, temozolomide, temsirolimus, teniposide, thapsigargin, thioguanine, thrombospondin-1, tiazofurin, topotecan, trapoxin, treosulfan, trichostatin A, trimetrexate, triplatin tetranitrate, trofosfamide, tumor necrosis factor, valproic acid, vemurafenib, VER49009, verapamil, vertoporfin, vinblastine, vincristine, vindesine, vinorelbine vitamin D3, VX-680, zactima, ZK-EPO, zorubicin, trastuzumab, cetuximab, lambrolizumab, nivolumab or any combination thereof.

The disclosed compounds may be included in kits comprising the compound [e.g., one or more compounds of formula (I)], a systemic or topical composition described above, or both; and information, instructions, or both that use of the kit will provide treatment for medical conditions in mammals (particularly humans). The information and instructions may be in the form of words, pictures, or both, and the like. In addition or in the alternative, the kit may include the medicament, a composition, or both; and information, instructions, or both, regarding methods of application of medicament, or of composition, preferably with the benefit of treating or preventing medical conditions in mammals (e.g., humans).

### 3. COMPOUNDS

In one aspect, disclosed is a compound of formula (I):

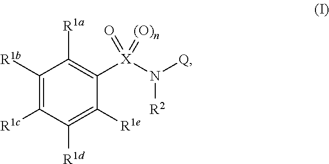

or a pharmaceutically acceptable salt thereof; wherein one of R^{1a}, R^{1b}, R^{1c}, R^{1d} and R^{1e} is OR⁴ or NR⁸—SO₂—R⁹, and the remaining are independently selected from hydrogen, halogen, nitro, alkyl, cyano, haloalkyl, alkoxyalkyl, heteroalkyl, alkenyl, alkynyl, heterocycle, carboxyl, heterocyclealkyl, OR⁴, SR⁵, NR⁶R⁷ and NR⁸—SO₂—R⁹; or R^{1b} and R^{1c}, R^{1c} and R^{1d}, or R^{1d} and R^{1e} together form a six-membered aromatic ring; X is C or S; n is 0 or 1; R² is selected from hydrogen, —C(O)-alkyl, —C(O)-alkenyl, —C(O)-alkoxyalkyl, —C(O)-heteroalkyl, —C(O)— heteroaryl, —C(O)—O-heteroalkyl, —C(O)—O-heteroaryl, —C(O)—O-alkyl, —C(O)—O-alkenyl, and —C(O)—O-alkoxyalkyl, or R² and R^{1e} together form a ring; Q is heteroaryl, with 0-5 substituents independently selected from hydrogen, halogen, nitro, alkyl, cyano, haloalkyl, alkoxyalkyl, heteroalkyl, alkenyl, alkynyl, heterocycle, carboxyl, heterocyclealkyl, OR⁴, SR⁵, NR⁶R⁷, and NR⁸—SO₂—R⁹; R⁴ is selected from hydrogen, —C(O)-alkyl, —C(O)-alkenyl, —C(O)-alkoxyalkyl, —C(O)-heteroalkyl, —C(O)-heteroaryl, —C(O)—O-heteroalkyl, —C(O)—O-heteroaryl, —C(O)—O— alkyl, —C(O)—O-alkenyl, and —C(O)—O-alkoxyalkyl —C(O)—NH-alkyl, and —C(O)-heterocycle; R⁵, R⁶ and R⁷ are each independently selected from hydrogen, alkyl, —C(O)-alkyl, —C(O)— alkenyl, —C(O)—O-alkyl, —C(O)—O-alkenyl, —C(O)-alkoxyalkyl, —C(O)—NH-alkyl, —C(O)-heterocycle, alkenyl, alkynyl, and heteroalkyl; R⁸ is selected from hydrogen and alkyl; and R⁹ is selected from hydrogen, alkyl, aryl, heteroaryl, arylalkyl, heterocycle, and heteroarylalkyl.

In certain embodiments, one of R^{1a}, R^{1b}, R^{1c}, R^{1d} and R^{1e} is OR⁴ or NR—SO₂—R⁹, and the remaining are independently selected from hydrogen, halogen, and OR⁴; R⁴ is selected from hydrogen, —C(O)-alkyl. —C(O)-alkenyl, —C(O)—O-alkyl, —C(O)—O-alkenyl, —C(O)— alkoxyalkyl, —C(O)—NH-alkyl, and —C(O)-heterocycle; R⁸ is selected from hydrogen and alkyl; and R⁹ is selected from alkyl, aryl, and heteroaryl.

In certain embodiments, one of R^{1a} and R^{1e} is OR⁴ or NR⁸—SO₂—R⁹; R^{1b}, R^{1c}, and R^{1d} are independently selected from hydrogen, halogen, and OR⁴; R⁴ is selected from hydrogen, —C(O)-alkyl, —C(O)-alkenyl, —C(O)—O-alkyl, —C(O)—O-alkenyl, —C(O)-alkoxyalkyl, —C(O)—NH-alkyl, and —C(O)-heterocycle; R is selected from hydrogen and alkyl; and R⁹ is selected from alkyl, aryl, and heteroaryl.

In certain embodiments, R^{1a} is OR⁴; R^{1b}, R^{1c} and R^{1e} are hydrogen; R^{1d} is hydrogen or halogen; and R⁴ is hydrogen, —C(O)-alkyl, —C(O)-alkenyl, or —C(O)-alkoxyalkyl.

In certain embodiments, R^{1a} is OR⁴; R^{1b}, Rio and Rio are hydrogen; R^{1d} is hydrogen or halogen; and R⁴ is hydrogen, —C(O)-alkyl or —C(O)-alkenyl.

In certain embodiments, R^{1a} is OR⁴; R^{1b}, R^{1c} and R^{1e} are hydrogen; R^{1d} is hydrogen or halogen; and R⁴ is hydrogen or alkyl.

In certain embodiments, R² is selected from hydrogen, C(O)-alkyl, —C(O)-alkenyl, —C(O)-alkoxyalkyl, —C(O)—O-alkyl, —C(O)—O-alkenyl, and —C(O)—O-alkoxyalkyl.

In certain embodiments, R² is selected from hydrogen, —C(O)-alkyl, —C(O)— alkoxyalkyl, —C(O)—O-alkyl and —C(O)—O-alkoxyalkyl.

In certain embodiments, R² is selected from hydrogen and —C(O)-alkyl.

In certain embodiments, R² and R^{1e} together form a 5 to 8-membered ring. In certain embodiments, R² and R^{1e} together form a 5-membered ring. In certain embodiments, R² and R^{1e} together form a 6-membered ring. In certain embodiments, R² and R^{1e} together form a 7-membered ring. In certain embodiments, R² and R^{1e} together form an 8-membered ring.

In certain embodiments, Q is a 5 or 6 membered heteroaryl.

In certain embodiments, Q is a 6 membered heteroaryl.

In certain embodiments, Q is a 5 membered heteroaryl.

In certain embodiments, Q is pyridinyl (including pyridin-2-yl, pyridin-3-yl, pyridin-4-yl), pyrimidinyl, pyridazinyl, pyrazinyl, thiazolyl, oxazolyl, pyrazolyl, imidazolyl, thiadiazolyl, oxadiazolyl, pyrrolyl, thienyl, furanyl, isoxazolyl, isothiazolyl, triazolyl, or tetrazolyl.

In certain embodiments, Q is pyridinyl (including pyridin-2-yl, pyridin-3-yl, pyridin-4-yl), pyrimidinyl, pyridazinyl, or pyrazinyl.

In certain embodiments, Q is thiazolyl, oxazolyl, pyrazolyl, imidazolyl, thiadiazolyl, oxadiazolyl, pyrrolyl, thienyl, furanyl, isoxazolyl, isothiazolyl, triazolyl, or tetrazolyl.

In certain embodiments, Q is substituted with 0, 1 or 2 substituents independently selected from the group consisting of halogen, nitro, alkyl, cyano, haloalkyl, alkoxyalkyl, heteroalkyl, alkenyl, alkynyl, heterocycle, carboxyl, heterocyclealkyl, OR⁴, SR⁵, NR⁶R⁷, and NR⁸—SO₂—R⁹. In certain embodiments, Q is substituted with 1 or 2 substituents independently selected from the group consisting of halogen, nitrogen and haloalkyl.

In certain embodiments, X is C and n is 0.

In certain embodiments, X is S and n is 1.

In certain embodiments, R^{1a} is OR⁴; R^{1b}, R^{1c} and R^{1e} are hydrogen; R^{1d} is hydrogen or halogen; R² is hydrogen; R⁴ is selected from C(O)-alkyl, —C(O)-alkenyl, —C(O)— alkoxyalkyl, —C(O)—O-alkyl, —C(O)—O-alkenyl, and —C(O)—O-alkoxyalkyl; Q is a 5 or 6 membered heteroaryl; X is C; and n is 0.

In certain embodiments, R^{1a} is OR⁴; R^{1b}, R^{1c} and R^{1e} are hydrogen; R^{1d} is hydrogen or halogen; R² is hydrogen; R⁴ is hydrogen or alkyl; Q is a 5 or 6 membered heteroaryl; X is C: and n is 0.

In certain embodiments, R^{1a} is OR⁴; R^{1b}, R^{1c} and R^{1e} are hydrogen; R^{1d} is hydrogen or halogen; R² is hydrogen; R⁴ is selected from C(O)-alkyl, —C(O)-alkenyl, —C(O)— alkoxyalkyl, —C(O)—O-alkyl, —C(O)—O-alkenyl, and —C(O)—O-alkoxyalkyl; Q is a 5 or 6 membered heteroaryl; X is S; and n is 1.

In certain embodiments, R^{1a} is OR⁴; R^{1b}, R^{1c} and R^{1e} are hydrogen; R^{1d} is hydrogen or halogen; R² is hydrogen; R⁴ is hydrogen or alkyl; Q is a 5 or 6 membered heteroaryl; X is S; and n is 1.

In certain embodiments. R^{1a} is OR⁴; R^{1b}, R^{1c} and R^{1e} are hydrogen; R^{1d} is hydrogen or halogen; R² is hydrogen; R⁴ is selected from C(O)-alkyl, —C(O)-alkenyl, —C(O)— alkoxyalkyl, —C(O)—O-alkyl, —C(O)—O-alkenyl, and —C(O)—O-alkoxyalkyl; Q is a 5 membered heteroaryl; X is C; and n is 0.

In certain embodiments, R^{1a} is OR⁴; R^{1b}, R^{1c} and R^{1e} are hydrogen; R^{1d} is hydrogen or halogen; R² is hydrogen; R⁴ is hydrogen or alkyl; Q is a 5 membered heteroaryl; X is C; and n is 0.

In certain embodiments, R^{1a} is OR⁴; R^{1b}, R^{1c} and R^{1e} are hydrogen; R^{1d} is hydrogen or halogen; R² is hydrogen; R⁴ is selected from C(O)-alkyl, —C(O)-alkenyl, —C(O)— alkoxyalkyl, —C(O)—O-alkyl, —C(O)—O-alkenyl, and —C(O)—O-alkoxyalkyl; Q is a 5 membered heteroaryl; X is S; and n is 1.

In certain embodiments. R^{1a} is OR⁴; R^{1b}, R^{1c} and R^{1e} are hydrogen; R^{1d} is hydrogen or halogen; R² is hydrogen; R⁴ is hydrogen or alkyl; Q is a 5 membered heteroaryl; X is S; and n is 1.

In some embodiments, R² and R^{1e} together form a ring.

Representative compounds of formula (I) include, but are not limited to:
5-chloro-N-(3-chloropyridin-4-yl)-2-hydroxybenzamide;
5-chloro-2-hydroxy-N-(5-nitropyridin-2-yl)benzamide;
5-chloro-2-hydroxy-N-(5-(trifluoromethyl)pyridin-2-yl)benzamide;
5-chloro-N-(3-chloro-5-(trifluoromethyl)pyridin-2-yl)-2-hydroxybenzamide;
5-chloro-2-hydroxy-N-(5-nitrothiazol-2-yl)benzamide;
2-hydroxy-N-(5-nitrothiazol-2-yl)benzamide; and
2-((5-nitrothiazol-2-yl)carbamoyl)phenyl acetate,

or a pharmaceutically acceptable salt thereof.

Representative compounds of formula (I) may also include, but are not limited to:
5-chloro-N-(4-chloro-6-(trifluoromethyl)pyridin-3-yl)-2-hydroxybenzamide;
5-chloro-N-(4-chloro-6-nitropyridin-3-yl)-2-hydroxybenzamide;
5-chloro-N-(2-chloro-6-nitropyridin-3-yl)-2-hydroxybenzamide;
5-chloro-N-(2-chloro-6-(trifluoromethyl)pyridin-3-yl)-2-hydroxybenzamide;
5-chloro-2-hydroxy-N-(6-(trifluoromethyl)pyridazin-3-yl)benzamide;
5-chloro-2-hydroxy-N-(6-nitropyridazin-3-yl)benzamide;
5-chloro-2-hydroxy-N-(6-(trifluoromethyl)pyridin-3-yl)benzamide;
5-chloro-2-hydroxy-N-(6-nitropyridin-3-yl)benzamide;
5-chloro-N-(4-chloro-6-(trifluoromethyl)pyridazin-3-yl)-2-hydroxybenzamide
5-chloro-N-(4-chloro-6-nitropyridazin-3-yl)-2-hydroxybenzamide;
5-chloro-N-(4-chloro-2-(trifluoromethyl)pyrimidin-5-yl)-2-hydroxybenzamide;
5-chloro-N-(4-chloro-2-nitropyrimidin-5-yl)-2-hydroxybenzamide;
5-chloro-2-hydroxy-N-(2-(trifluoromethyl)pyrimidin-5-yl)benzamide;
5-chloro-2-hydroxy-N-(2-nitropyrimidin-5-yl)benzamide;
5-chloro-N-(5-chloro-6-(trifluoromethyl)pyridin-3-yl)-2-hydroxybenzamide;
5-chloro-N-(5-chloro-6-nitropyridin-3-yl)-2-hydroxybenzamide;
5-chloro-N-(5-chloro-6-(trifluoromethyl)pyridazin-3-yl)-2-hydroxybenzamide; and
5-chloro-N-(5-chloro-6-nitropyridazin-3-yl)-2-hydroxybenzamide,

or a pharmaceutically acceptable salt thereof.

Representative compounds of formula (I) may also include, but are not limited to:
4-chloro-2-((5-chloro-6-nitropyridin-3-yl)carbamoyl)phenyl octanoate;
4-chloro-2-((5-chloro-6-nitropyridazin-3-yl)carbamoyl)phenyl octanoate;
4-chloro-2-((4-chloro-6-(trifluoromethyl)pyridin-3-yl)carbamoyl)phenyl octanoate;
4-chloro-2-((4-chloro-6-nitropyridin-3-yl)carbamoyl)phenyl octanoate;
4-chloro-2-((2-chloro-6-(trifluoromethyl)pyridin-3-yl)carbamoyl)phenyl octanoate;
4-chloro-2-((2-chloro-6-nitropyridin-3-yl)carbamoyl)phenyl octanoate;
4-chloro-2-((6-(trifluoromethyl)pyridazin-3-yl)carbamoyl)phenyl octanoate;
4-chloro-2-((6-nitropyridazin-3-yl)carbamoyl)phenyl octanoate;
4-chloro-2-((4-chloro-2-(trifluoromethyl)pyrimidin-5-yl)carbamoyl)phenyl octanoate;
4-chloro-2-((4-chloro-6-nitropyridazin-3-yl)carbamoyl)phenyl octanoate;
4-chloro-2-((2-(trifluoromethyl)pyrimidin-5-yl)carbamoyl)phenyl octanoate;
4-chloro-2-((2-nitropyrimidin-5-yl)carbamoyl)phenyl octanoate;
4-chloro-2-((6-(trifluoromethyl)pyridin-3-yl)carbamoyl)phenyl octanoate; and
6-chloro-3-(2-chloro-6-(trifluoromethyl)pyridin-3-yl)-2H-benzo[e][1,3]oxazine-2,4(3H)-dione,

or a pharmaceutically acceptable salt thereof.

Compound names are assigned by using Struct=Name naming algorithm as part of CHEMDRAW® ULTRA v. 12.0.

The compound may exist as a stereoisomer wherein asymmetric or chiral centers are present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Fumiss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

It should be understood that the compound may possess tautomeric forms, as well as geometric isomers, and that these also constitute an aspect of the invention.

The present invention also includes an isotopically-labeled compound, which is identical to those recited in formula (I), but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

A. Inhibition of Wnt Signaling

The disclosed compounds may act or function as inhibitors of the Wnt/Frizzled signaling pathway. The compounds may promote the anti-proliferation of cancer cells even in the presence of Wnt/Frizzled signaling dysfunction.

Compounds of formula (I) can inhibit Wnt-3A-stimulated signaling with an IC₅₀ ranging from about 1 nM to about 30 μM. The compounds may have an IC₅₀ of about 30 μM, about 29 μM, about 28 μM, about 27 μM, about 26 μM, about 25 μM, about 24 μM, about 23 μM, about 22 μM, about 21 μM, about 20 μM, about 19 μM, about 18 μM, about 17 μM, about 16 μM, about 15 μM, about 14 μM, about 13 μM, about 12 μM, about 11 μM, about 10 μM, about 9 μM, about 8 μM, about 7 μM, about 6 μM, about 5 μM, about 4 μM, about 3 μM, about 2 μM, about 1 μM, about 950 nM, about 900 nM, about 850 nM, about 800 nM, about 850 nM, about 800 nM, about 750 nM, about 700 nM, about 650 nM, about 600 nM, about 550 nM, about 500 nM, about 450 nM, about 400 nM, about 350 nM, about 300 nM, about 250 nM, about 200 nM, about 150 nM, about 100 nM, about 50 nM, about 10 nM, about 5 nM, or about 1 nM. Compounds of formula (I) can inhibit Wnt-3A-stimulated signaling with an IC₅₀ of less than 30 μM, less than 29 μM, less than 28 μM, less than 27 μM, less than 26 μM, less than 25 μM, less than 24 μM, less than 23 μM, less than 22 μM, less than 21 μM, less than 20 μM, less than 19 μM, less than 18 μM, less than 17 μM, less than 16 μM, less than 15 μM, less than 14 μM, less than 13 μM, less than 12 μM, less than 11 μM, less than 10 μM, less than 9 μM, less than 8 μM, less than 7 μM, less than 6 μM, less than 5 μM, less than 4 μM, less than 3 μM, less than 2 μM, less than 1 μM, less than 950 nM, less than 900 nM, less than 850 nM, less than 800 nM, less than 850 nM, less than 800 nM, less than 750 nM, less than 700 nM, less than 650 nM, less than 600 nM, less than 550 nM, less than 500 nM, less than 450 nM, less than 400 nM, less than 350 nM, less than 300 nM, less than 250 nM, less than 200 nM, less than 150 nM, less than 100 nM, less than 50 nM, less than 10 nM, less than 5 nM, or less than 1 nM.

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

B. General Synthesis

1. Compound of Formula (I)

Compounds of formula (I) may be prepared by synthetic processes or by metabolic processes. Preparation of the compounds by metabolic processes includes those occurring in the human or animal body (in vivo) or processes occurring in vitro.

Compounds of formula (I), wherein the groups R^{1a}, R^{1b}, R^{1c}, R^{1d}, R^{1e}, R², and Q have the meanings as set forth in the Summary of the Invention section and X is S and is 1, can be synthesized as shown in Scheme 1.

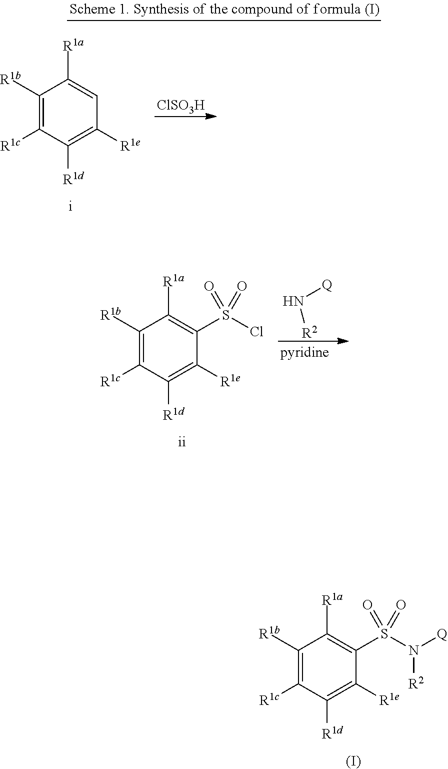

As shown in Scheme 1, intermediate ii, wherein R^{1a}, R^{1b}, R^{1c}, R^{1d} and R^{1e} are as defined in the Summary of the Invention, can be prepared from the substituted benzene, i, and chlorosulfonic acid. Treatment of ii with an amine, wherein R² and Q are as defined in the Summary of the Invention, can provide the compound of formula (I).

Compounds of formula (1), wherein the groups R^{1a}, R^{1b}, R^{1c}, R^{1d}, R^{1e}, R², and Q have the meanings as set forth in the Summary of the Invention section and X is C and is 0, can be synthesized as shown in Scheme 1.

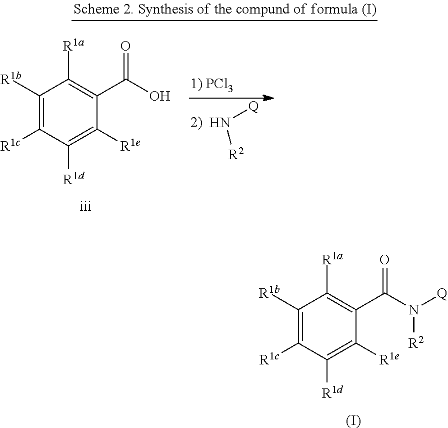

Benzoic acid iii, wherein R^{1a}, R^{1b}, R^{1c}, R^{1d} and R^{1e} are as defined in the Summary of the Invention, can be transformed to the acid chloride by treatment with PCl₃. The acid chloride intermediate can be treated with an amine, wherein R² and Q are as defined in the Summary of the Invention, to provide the compound of formula (I).

Employing analogous synthetic methods and the syntheses provided in the Examples, the remaining compounds of the disclosure may be obtained.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Fumrniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Optimum reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in P G M Wuts and T W Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### 4. PHARMACEUTICAL COMPOSITIONS

The disclosed compounds may be incorporated into pharmaceutical compositions suitable for administration to a subject (such as a patient, which may be a human or non-human).

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention [e.g., a compound of formula (I)] are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of a compound of formula (I), may be about 1 mg/kg to about 1000 mg/kg, about 5 mg/kg to about 950 mg/kg, about 10 mg/kg to about 900 mg/kg, about 15 mg/kg to about 850 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts and solvates may be formulated for administration by, for example, solid dosing, eyedrop, in a topical oil-based formulation, injection, inhalation (either through the mouth or the nose), implants, or oral, buccal, parenteral, or rectal administration. Techniques and formulations may generally be found in “Remington's Pharmaceutical Sciences”, (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmelose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, Pa.) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of active [e.g., compound of formula (I)] and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmelose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound [e.g., a compound of formula (I)], and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT coatings (available from Rohm & Haas G.M.B.H. of Darmstadt, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound [e.g., a compound of formula (I)], and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

The compounds and processes of the invention will be better understood by reference to the following examples, which are intended as an illustration of and not a limitation upon the scope of the invention.

### 5. EXAMPLES

### Example 1. Compound Synthesis

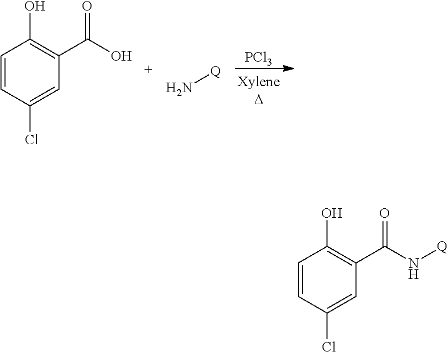

General Method:

To a 100 mL flask equipped with a reflux condenser was added 5-chloro-2-hydroxybenzoic acid (1 equiv.), the aminoheteroaryl derivative NH₂-Q (1 equiv.), and dry xylenes (stored over 3A molecular sieves, 40 mL per gram of 5-chloro-2-hydroxybenzoic acid) under an atmosphere of argon. The mixture was heated to reflux, and PCl₃ (0.4 equiv.) was added rapidly via syringe. The mixture was heated at reflux for 1 hour and cooled to room temperature. Water (40 mL per gram of 5-chloro-2-hydroxybenzoic acid) was added and the resultant heterogeneous mixture stirred rapidly for 1 hour. Saturated sodium bicarbonate was added to a final pH of 3-4, and the mixture stirred rapidly overnight. The solids were filtered and washed sequentially with water, toluene and hexane. Samples were analyzed by NMR, HPLC/mass spectrometry and TLC. Purification by crystallization or column chromatography on silica gel was performed when purity was less than 95% by LC. HPLC/MS was accomplished using an Agilent spectrometer—6310 Ion trap. Mass ions (m/z) detected in positive ionization mode are M+; in negative ionization mode, mass ions (m/z) are M−.

The following compounds were synthesized employing the general method above:

| No. | NH₂—Q | Compound | MS (m/z) |
|---|---|---|---|
| 1 | 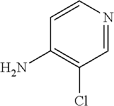 | 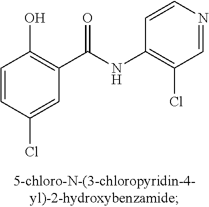 [SMILES: O=C(Nc1ccncc1Cl)c1cc(Cl)ccc1O] | ES(+) = 283, 285 (M + 1) |
| 2 | 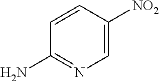 | 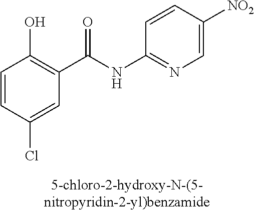 [SMILES: O=C(Nc1ccc([N+](=O)[O-])cn1)c1cc(Cl)ccc1O] | ES(−) = 292 (M − 1) |
| 3 | 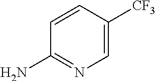 | 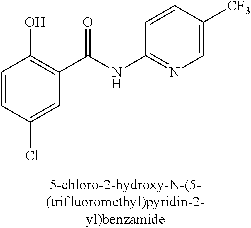 [SMILES: O=C(Nc1ccc(C(F)(F)F)cn1)c1cc(Cl)ccc1O] | ES(−) = 315 (M − 1) |
| 4 | 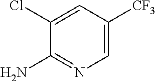 | 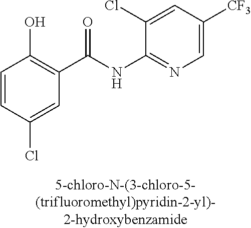 [SMILES: O=C(Nc1ncc(C(F)(F)F)cc1Cl)c1cc(Cl)ccc1O] | ES(−) = 349 (M − 1) |
| 5 | 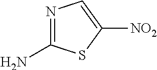 | 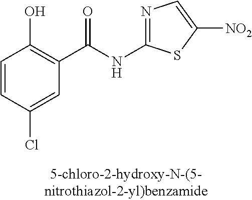 [SMILES: O=C(Nc1ncc([N+](=O)[O-])s1)c1cc(Cl)ccc1O] | ES(−) = 298 (M − 1) |

The following known compounds were purchased from commercial sources:

| compound | Structure and name |
|---|---|
| A | 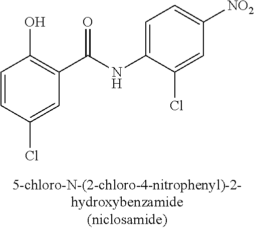 [SMILES: O=C(Nc1ccc([N+](=O)[O-])cc1Cl)c1cc(Cl)ccc1O] |
| B | 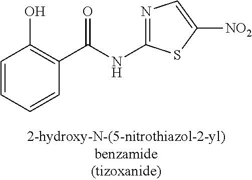 [SMILES: O=C(Nc1ncc([N+](=O)[O-])s1)c1ccccc1O] |
| C | 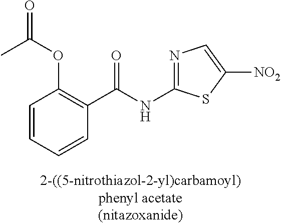 [SMILES: CC(=O)Oc1ccccc1C(=O)Nc1ncc([N+](=O)[O-])s1] |

### Example 2. Biological Activity

A. TOPFlash Reporter Assay

Wnt-3A conditioned medium was prepared using L WNT-3A cells (ATCC(CRL-2647™) purchased from ATCC. Conditioned medium was obtained following published protocols (http://www.atcc.org/Products/All/CRL-2647.aspx#culturemethod) (Chen, M. Y.; Wang, J. B.; Lu, J. Y.; Bond, M. C.; Ren, X. R.; Lyerly, H. K.; Barak, L. S.; Chen, W. Biochemistry 2009, 48, 10267). HEK293 cells were stably transfected with p8×TOPFlash, Renilla luciferase plasmid pRL-TK (Promega), and pLKO.1 as previously published (Chen, M. Y.; Wang, J. B.; Lu, J. Y.; Bond, M. C.; Ren, X. R.; Lyerly, H. K.; Barak, L. S.; Chen, W. Biochemistry 2009, 48, 10267). Stably transfected cells were seeded in 100 μl of cell growth medium/well in 96-well plates at 100% confluency. Fifty microliters of Wnt-3A conditioned medium containing the chemical compounds to be tested or DMSO was added to each well. After an 8 hour treatment, the cells were washed once with PBS and lysed with 55 μl of Passive Lysis Buffer supplied in the Dual-Luciferase Reporter Assay kit (Promega, Madison, Wis.). Twenty-five microliters of cell lysate was used for measuring luciferase activity in a 96-well plate reader (FluoStar Optima, BMG Labtech, Chicago, Ill.). Data were fit using GraphPad Prism (mean±SEM, n>=2).

B. Results and Discussion of In Vitro Biological Activity Data

The results of the Topflash Reporter assays are shown in Table 1. The data in Table 1 demonstrates that the disclosed compounds are inhibitors of the Wnt/Frizzled signaling pathway. Data is from a single experiment unless otherwise noted. Data that is an average of two experiments is noted as “n=2” while data that is an average of three or more experiments is presented as the average plus or minus the standard error of the mean.

TABLE 1

| | Inhibition Wnt/β- |
|---|---|
| | catenin transcription |
| Compound | TopFlash IC50 (μM) |
| A | 0.49 |
| B | >12 |
| C | 11.4 |
| 1 | 80% inhibition at 10 μM |
| 2 | 75% inhibition at 2 μM |
| 3 | 1.67 |
| 4 | 17.4 |
| 5 | 8.8 |

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A method of treating a disease associated with dysregulation of the Wnt/Frizzled signaling pathway in a subject in need thereof, the method comprising
administering to the subject an effective amount of a compound of formula (I), or a pharmaceutically acceptable salt thereof, 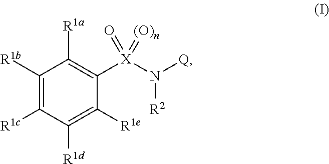
wherein,
one of R^{1a}, R^{1b}, R^{1c}, R^{1d} and R^{1e} is OR⁴ or NR⁸—SO₂—R⁹, and the remaining are independently selected from hydrogen, halogen, nitro, alkyl, cyano, haloalkyl, alkoxyalkyl, heteroalkyl, alkenyl, alkynyl, heterocycle, carboxyl, heterocyclealkyl, OR⁴, SR⁵, NR⁶R⁷ and NR⁸—SO₂—R⁹; or R^{1b} and R^{1c}, R^{1c} and R^{1d}, or R^{1d} and R^{1e} together form a six-membered aromatic ring;
X is C or S;
n is 0 or 1;
R² is selected from hydrogen, —C(O)-alkyl, —C(O)-alkenyl, —C(O)-alkoxyalkyl, —C(O)— heteroalkyl, —C(O)-heteroaryl, —C(O)—O-heteroalkyl, —C(O)—O-heteroaryl, —C(O)—O-alkyl, —C(O)—O— alkenyl, and —C(O)—O-alkoxyalkyl, or R² and R^{1e} together form a ring;
Q is heteroaryl, with 0-5 substituents independently selected from hydrogen, halogen, nitro, alkyl, cyano, haloalkyl, alkoxyalkyl, heteroalkyl, alkenyl, alkynyl, heterocycle, carboxyl, heterocyclealkyl, OR⁴, SR⁵, NR⁶R⁷, and NR⁸—SO₂—R⁹;
R⁴ is selected from hydrogen, —C(O)-alkyl, —C(O)-alkenyl, —C(O)-alkoxyalkyl, —C(O)-heteroalkyl, —C(O)-heteroaryl, —C(O)—O-heteroalkyl, —C(O)—O-heteroaryl, —C(O)—O-alkyl, —C(O)—O-alkenyl, and —C(O)—O-alkoxyalkyl —C(O)—NH-alkyl, and —C(O)-heterocycle;
R⁵, R⁶ and R⁷ are each independently selected from hydrogen, alkyl, —C(O)-alkyl, —C(O)-alkoxyalkyl, alkenyl, alkynyl, and heteroalkyl;
R⁸ is selected from hydrogen and alkyl; and
R⁹ is selected from hydrogen, alkyl, aryl, heteroaryl, arylalkyl, heterocycle, and heteroarylalkyl.

2. The method of claim 1, wherein
Q is a 5 or 6 membered heteroaryl.

3. The method of claim 1, wherein
X is C; and
n is 0.

4. The method of claim 1, wherein
X is S; and
n is 1.

5. The method of claim 1, wherein
Q is a 5 membered heteroaryl.

6. The method of claim 1, wherein
R^{1a} is OR⁴;
R^{1b}, R^{1c} and R^{1e} are hydrogen; and
R^{1d} is hydrogen or halogen.

7. The method of claim 1, wherein the compound of formula (I) is selected from the group consisting of:
5-chloro-N-(3-chloropyridin-4-yl)-2-hydroxybenzamide;
5-chloro-2-hydroxy-N-(5-nitropyridin-2-yl)benzamide;
5-chloro-2-hydroxy-N-(5-(trifluoromethyl)pyridin-2-yl)benzamide;
5-chloro-N-(3-chloro-5-(trifluoromethyl)pyridin-2-yl)-2-hydroxybenzamide;
5-chloro-2-hydroxy-N-(5-nitrothiazol-2-yl)benzamide;
2-hydroxy-N-(5-nitrothiazol-2-yl)benzamide; and
2-((5-nitrothiazol-2-yl)carbamoyl)phenyl acetate,
or a pharmaceutically acceptable salt thereof.

8. The method of claim 1, wherein the disease is cancer.

9. The method of claim 1, wherein the subject has at least one inactivating mutation of the Adenomatous Polyposis Coli (APC) gene.

10. The method of claim 1, wherein the subject has at least one mutation of the β-catenin gene or overexpression of the β-catenin protein, or a combination thereof.

11. The method of claim 1, wherein the subject has overexpression of Wnt ligands.

12. The method of claim 8, wherein the cancer is colorectal cancer, breast cancer, melanoma, prostate cancer, lung cancer, ovarian cancer, esophageal cancer, glioblastoma, multiple myeloma, mantle cell lymphoma, liver cancer, leukemia, acute myelogenous leukemia, or a combination thereof.

13. The method of claim 1, wherein the disease is nonalcoholic steatohepatitis or nonalcoholic fatty liver disease.

14. A method of treating cancer in a subject in need thereof, the method comprising
identifying a subject with dysregulated Wnt/Frizzled signaling pathway; and
administering to the subject with dysregulated Wnt/Frizzled signaling pathway an effective amount of a compound of formula (I), or a pharmaceutically acceptable salt thereof, 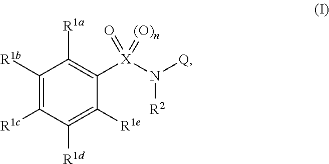
wherein,
one of R^{1a}, R^{1b}, R^{1c}, R^{1d} and R^{1e} is OR⁴ or NR⁸—SO₂—R⁹, and the remaining are independently selected from hydrogen, halogen, nitro, alkyl, cyano, haloalkyl, alkoxyalkyl, heteroalkyl, alkenyl, alkynyl, heterocycle, carboxyl, heterocyclealkyl, OR⁴, SR⁵, NR⁶R⁷ and NR⁸—SO₂—R⁹; or R^{1b} and R^{1c}, R^{1c} and R^{1d}, or R^{1d} and R^{1e} together form a six-membered aromatic ring;
X is C or S;
n is 0 or 1;
R² is selected from hydrogen, —C(O)-alkyl, —C(O)-alkenyl, —C(O)-alkoxyalkyl, —C(O)— heteroalkyl, —C(O)-heteroaryl, —C(O)—O-heteroalkyl, —C(O)—O-heteroaryl, —C(O)—O-alkyl, —C(O)—O— alkenyl, and —C(O)—O-alkoxyalkyl, or R² and R^{1e} together form a ring;
Q is heteroaryl, with 0-5 substituents independently selected from hydrogen, halogen, nitro, alkyl, cyano, haloalkyl, alkoxyalkyl, heteroalkyl, alkenyl, alkynyl, heterocycle, carboxyl, heterocyclealkyl, OR⁴, SR⁵, NR⁶R⁷, and NR⁸—SO₂—R⁹;
R⁴ is selected from hydrogen, —C(O)-alkyl, —C(O)-alkenyl, —C(O)-alkoxyalkyl, —C(O)-heteroalkyl, —C(O)-heteroaryl, —C(O)—O-heteroalkyl, —C(O)—O-heteroaryl, —C(O)—O-alkyl, —C(O)—O-alkenyl, and —C(O)—O-alkoxyalkyl —C(O)—NH-alkyl, and —C(O)-heterocycle;
R⁵, R⁶ and R⁷ are each independently selected from hydrogen, alkyl, —C(O)-alkyl, —C(O)-alkoxyalkyl, alkenyl, alkynyl, and heteroalkyl;
R⁸ is selected from hydrogen and alkyl; and
R⁹ is selected from hydrogen, alkyl, aryl, heteroaryl, arylalkyl, heterocycle, and heteroarylalkyl.

15.-19. (canceled)

20. The method of claim 14, wherein the compound of formula (I) is selected from the group consisting of:
5-chloro-N-(3-chloropyridin-4-yl)-2-hydroxybenzamide;
5-chloro-2-hydroxy-N-(5-nitropyridin-2-yl)benzamide;
5-chloro-2-hydroxy-N-(5-(trifluoromethyl)pyridin-2-yl)benzamide;
5-chloro-N-(3-chloro-5-(trifluoromethyl)pyridin-2-yl)-2-hydroxybenzamide;
5-chloro-2-hydroxy-N-(5-nitrothiazol-2-yl)benzamide;
2-hydroxy-N-(5-nitrothiazol-2-yl)benzamide; and
2-((5-nitrothiazol-2-yl)carbamoyl)phenyl acetate,
or a pharmaceutically acceptable salt thereof.

21.-23. (canceled)

24. The method of claim 1, wherein the cancer is colorectal cancer, breast cancer, melanoma, prostate cancer, lung cancer, ovarian cancer, esophageal cancer, glioblastoma, multiple myeloma, mantle cell lymphoma, liver cancer, leukemia, acute myelogenous leukemia, or a combination thereof.

25. A method of modulating the Wnt/Frizzled signaling pathway in a subject, the method comprising
administering to the subject an effective amount of a compound of formula (I), or a pharmaceutically acceptable salt thereof, 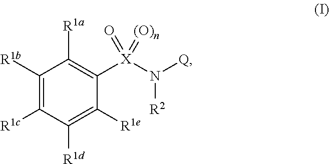
wherein,
one of R^{1a}, R^{1b}, R^{1c}, R^{1d} and R^{1e} is OR⁴ or NR⁸—SO₂—R⁹, and the remaining are independently selected from hydrogen, halogen, nitro, alkyl, cyano, haloalkyl, alkoxyalkyl, heteroalkyl, alkenyl, alkynyl, heterocycle, carboxyl, heterocyclealkyl, OR⁴, SR⁵, NR⁶R⁷ and NR⁸—SO₂—R⁹; or R^{1b} and R^{1c}, R^{1c} and R^{1d}, or R^{1d} and R^{1e} together form a six-membered aromatic ring;
X is C or S;
n is 0 or 1;
R² is selected from hydrogen, —C(O)-alkyl, —C(O)-alkenyl, —C(O)-alkoxyalkyl, —C(O)— heteroalkyl, —C(O)-heteroaryl, —C(O)—O-heteroalkyl, —C(O)—O-heteroaryl, —C(O)—O-alkyl, —C(O)—O— alkenyl, and —C(O)—O-alkoxyalkyl, or R² and R^{1e} together form a ring;
Q is heteroaryl, with 0-5 substituents independently selected from hydrogen, halogen, nitro, alkyl, cyano, haloalkyl, alkoxyalkyl, heteroalkyl, alkenyl, alkynyl, heterocycle, carboxyl, heterocyclealkyl, OR⁴, SR⁵, NR⁶R⁷, and NR⁸—SO₂—R⁹;
R⁴ is selected from hydrogen, —C(O)-alkyl, —C(O)-alkenyl, —C(O)-alkoxyalkyl, —C(O)-heteroalkyl, —C(O)-heteroaryl, —C(O)—O-heteroalkyl, —C(O)—O-heteroaryl, —C(O)—O-alkyl, —C(O)—O-alkenyl, and —C(O)—O-alkoxyalkyl —C(O)—NH-alkyl, and —C(O)-heterocycle;
R⁵, R⁶ and R⁷ are each independently selected from hydrogen, alkyl, —C(O)-alkyl, —C(O)-alkoxyalkyl, alkenyl, alkynyl, and heteroalkyl;
R⁸ is selected from hydrogen and alkyl; and
R⁹ is selected from hydrogen, alkyl, aryl, heteroaryl, arylalkyl, heterocycle, and heteroarylalkyl.

26.-30. (canceled)

31. The method of claim 25, wherein the compound of formula (I) is selected from the group consisting of:
5-chloro-N-(3-chloropyridin-4-yl)-2-hydroxybenzamide;
5-chloro-2-hydroxy-N-(5-nitropyridin-2-yl)benzamide;
5-chloro-2-hydroxy-N-(5-(trifluoromethyl)pyridin-2-yl)benzamide;
5-chloro-N-(3-chloro-5-(trifluoromethyl)pyridin-2-yl)-2-hydroxybenzamide;
5-chloro-2-hydroxy-N-(5-nitrothiazol-2-yl)benzamide;
2-hydroxy-N-(5-nitrothiazol-2-yl)benzamide; and
2-((5-nitrothiazol-2-yl)carbamoyl)phenyl acetate,
or a pharmaceutically acceptable salt thereof.

32. A compound selected from the group consisting of:
5-chloro-N-(3-chloropyridin-4-yl)-2-hydroxybenzamide;
5-chloro-2-hydroxy-N-(5-nitropyridin-2-yl)benzamide;
5-chloro-2-hydroxy-N-(5-(trifluoromethyl)pyridin-2-yl)benzamide;
5-chloro-N-(3-chloro-5-(trifluoromethyl)pyridin-2-yl)-2-hydroxybenzamide; and
5-chloro-2-hydroxy-N-(5-nitrothiazol-2-yl)benzamide,
or a pharmaceutically acceptable salt thereof.

33. The method of claim 1, further comprising administering at least one of cisplatin, oxaliplatin, a kinase inhibitor, trastuzumab, cetuximab, panitumumab, lambrolizumab and nivolumab.
